(19) United States
(12) Patent Application Publication
(10) Pub. No.: US 2023/0214704 A1
(43) Pub. Date: Jul. 6, 2023

(54) MACHINE LEARNING FOR REAL-TIME MOTION CLASSIFICATION

(71) Applicant: MatrixCare, Inc., Bloomington, MN (US)

(72) Inventor: Nate SUKHTIPYAROGE, Bloomington, MN (US)

(21) Appl. No.: 17/566,492

(22) Filed: Dec. 30, 2021

(51) Int. Cl. G06N 20/00; G06F 1/16; G16H 40/20

(52) U.S. Cl. CPC G06N 20/00; G06F 1/163; G16H 40/20

(57) ABSTRACT

Techniques for improved machine learning are provided. Motion data collected during a first time by one or more wearable sensors of a user is received, and a patient associated with the motion data is identified. An action performed by the user is identified by processing the motion data using a machine learning model, and an event record indicating the action, the patient, and the user is generated.

### INTRODUCTION

Embodiments of the present disclosure relate to machine learning. More specifically, embodiments of the present disclosure relate to using machine learning to classify motion data.

In a wide variety of medical (and non-medical) settings, users are often expected to record or otherwise preserve indications of the actions they take, in order to preserve a concrete record of the tasks performed, when they were performed, who performed them, and the like. For example, nursing staff and other caregivers conventionally record their actions in patient charts. This process is often referred to as “charting.” Generally, the users chart any action or task performed, such as assisting a patient to eat, repositioning a patient, cleaning a wound, and the like. However, given the large number of tasks performed by each user, it is generally difficult to record all such tasks immediately after performing them (e.g., because the user must often continue to another task).

As a result, many users delay charting these actions until a later time (e.g., at the end of their work shift), resulting in significant inaccuracies and missed items (e.g., due to forgotten or misremembered tasks). Inaccuracies in these records can have a wide variety of long-reaching impacts.

Improved systems and techniques to automatically classify and record such tasks are needed.

### SUMMARY

According to one embodiment presented in this disclosure, a method of training machine learning models is provided. The method includes: receiving motion data collected during a first time by one or more wearable sensors of a user; identifying an action performed by the user during the first time by evaluating one or more event records indicating one or more prior actions performed by one or more users; labeling the motion data based on the action; and training a machine learning model, based on the labeled motion data, to identify user actions.

According to one embodiment presented in this disclosure, a method of classifying actions using machine learning models is provided. The method includes: receiving motion data collected during a first time by one or more wearable sensors of a user; identifying a patient associated with the motion data; identifying an action performed by the user by processing the motion data using a machine learning model; and generating an event record indicating the action, the patient, and the user.

The following description and the related drawings set forth in detail certain illustrative features of one or more embodiments.

### DESCRIPTION OF THE DRAWINGS

The appended figures depict certain aspects of the one or more embodiments and are therefore not to be considered limiting of the scope of this disclosure.

FIG. 1 depicts an example environment for training machine learning models to evaluate sensor data and classify motion.

FIG. 2 depicts an example workflow for generating labeled data to train machine learning models to classify motion.

FIG. 3 depicts an example environment for using machine learning models to classify sensor data.

FIG. 4 is a flow diagram depicting an example method for training machine learning models to classify motion data.

FIG. 5 is a flow diagram depicting an example method for generating labeled data to train machine learning models to classify motions.

FIG. 6 is a flow diagram depicting an example method for generating labeled data to train machine learning models to classify motions.

FIG. 7 is a flow diagram depicting an example method for refining machine learning models to predict actions.

FIG. 8 is a flow diagram depicting an example method for using trained machine learning models to evaluate sensor data and predict actions.

FIG. 9 is a flow diagram depicting an example method for evaluating motion data using machine learning models.

FIG. 10 is a flow diagram depicting an example method for identifying relevant user(s) for predicted actions.

FIG. 11 is a flow diagram depicting an example method for identifying relevant patient(s) for predicted actions.

FIG. 12 is a flow diagram depicting an example method for identifying relevant user(s) and/or patient(s) for predicted actions.

FIG. 13 is a flow diagram depicting an example method for refining machine learning models based on real-time motion classifications.

FIG. 14 is a flow diagram depicting an example method for training one or more machine learning models to identify user actions based on motion data.

FIG. 15 is a flow diagram depicting an example method for identifying user actions using machine learning.

FIG. 16 depicts an example computing device configured to perform various aspects of the present disclosure.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

Aspects of the present disclosure provide apparatuses, methods, processing systems, and computer-readable mediums for improved machine learning models for classifying motion data based on the underling action(s) being performed.

In embodiments, motion data from one or more wearable devices (e.g., wrist-mounted smart watches) can be collected and evaluated using one or more trained machine learning models in order to identify the action being performed by the wearer of the device(s). For example, based on prior training, the model(s) may determine what assistance the user is providing to a patient, such as transferring them to a seated position, treating a wound, assisting with eating, and the like. Though some examples discussed herein relate to identifying and classifying caregiver assistance given to patients or residents, embodiments of the present disclosure are readily applicable to a wide variety of implementations.

Generally, by training and using the machine learning model(s) to classify motion data, the system may be able to automatically record or chart user actions in order to significantly reduce manual effort, increase the accuracy of the records, and improve the overall system through creation of better data and improved results for the patients and users. Further, any downstream systems or models that rely on this event data can be significantly improved. For example, downstream machine learning systems that make predictions or inferences based on the performed actions (e.g., to track user status) will be greatly improved by more accurate and voluminous action records.

In an embodiment, the motion data is generally indicative of the motion of one or more parts of the user(s). For example, the motion data may include accelerometer data (e.g., three-axis accelerometer data) from wearable wrist-mounted sensors. Such data can generally be used to determine the orientation of the user's hands or arms, as well as the movement of the arms or hands (e.g., the directionality of movement, the speed and/or acceleration of the movement, and the like). Using machine learning, as discussed in more detail below, this movement can be used to identify or classify the user actions. For example, the motion and orientation of the user's hand when lifting a spoon to feed a resident differs from the motion and orientation used to lift a resident's leg or arm. By learning to identify and classify these motions, the system is able to automatically identify the actions being performed.

In at least one embodiment, in addition to such motion data, the system may also receive and evaluate other contextual data, such as proximity data indicating the user(s) and/or resident(s) in a given space during the action, data from sensor(s) in the space (e.g., pressure sensors or infrared sensors indicating where the user and/or patient are, such as whether they are moving from the bed towards the bathroom), and the like. This additional data may be used, in some embodiments, to provide more robust machine learning.

In some embodiments, in order to train the model(s), the system can automatically identify action(s) that one or more users performed, and retrieve the corresponding motion data from the user at the time the action was performed. For example, in a residential facility, the system may parse charts or other records to identify care actions (such as feeding a resident). For each such action, the system may determine the user that performed the action, as well as the context of the action (e.g., the time the action was performed, the resident that the user was assisting, and the like). Based on this action context, the system can retrieve motion data recorded at the time of the action being performed, and automatically label the motion data based on the action that was performed. This can allow the system to automatically label a vast amount of training data, enabling improved and more efficient training of the machine learning model(s).

In at least one embodiment, once the model(s) are trained, the system can automatically generate event or action records based on received motion data. For example, based on motion data (which may be streamed in real-time while performing the action, or transmitted after the action is completed) from the user's wearable sensor(s), the system may determine or infer which action(s) were performed. In some embodiments, the system can further automatically identify which resident(s) or patient(s) were being assisted.

In one such embodiment, the user (e.g., caregiver) can perform a check-in scan before they begin an action. For example, when entering a resident's room, the user may scan a device (such as a smartphone, a wrist-mounted wearable, an ID badge, and the like) at a reader near the entrance of the room (e.g., a Bluetooth device, an RFID device, an NFC device, and the like). Generally, the check-in device (e.g., a device affixed on or near a door) and the user device (e.g., the badge or wearable) may each be active devices, or one may be a passive device. For example, the check-in device may be an active device such as an RFID reader, or a passive device such as a barcode or QR code that is scanned by the user device. Similarly, the user device may be active, such as a Bluetooth transmitter/receiver, or a passive device such as a barcode or QR code that is read by the check-in device.

Using such check-in data, the system may determine where the action was performed (e.g., what room the user was entering or in), and thereby determine which patient(s) resides in or is otherwise associated with that location. This can allow the system to determine which patient(s) was being assisted by the user at any given time. This patient identification can then be included in the generated action or event record(s) reflecting what assistance was performed, which user performed it, and who was assisted.

In some embodiments, in addition to or instead of using a manual check-in process, the system can determine or infer the relevant patient(s) and/or user(s) using proximity sensors. In one such embodiment, the user device may be configured to automatically detect patient device(s) within defined distances (or vice versa). For example, the user device may use relatively short-range wireless communication protocols to detect nearby patient(s), and identify which patient is closest (e.g., based on signal strength of detected devices). It may then be inferred that this patient is being assisted during the action, and the system can update the generated record to indicate the patient.

In at least one embodiment, if multiple users assist in an action (e.g., turning a patient in bed, or helping a patient stand or sit), the system can identify the participating users (e.g., using proximity sensors as discussed above, and/or using check-in data). The system can then identify or select one of these users to serve as the “primary” user in the record, while the other users can be included as assisting users. This can allow the system to prevent recording the same action or assistance multiple times. That is, rather than mistakenly generate two records indicating that the patient was assisted twice, the system can generate a single record indicating that two users assisted the patient once.

In some examples discussed herein, the model(s) are generally trained to identify and classify caregiving actions. As used herein, caregiving actions can generally correspond to any action performed by a user (e.g., a caregiver, nurse, doctor, aide, and the like) to aid or assist a patient or resident (e.g., in a long-term care facility such as a nursing home). For example, the caregiving actions may include actions such as toileting (e.g., assisting the patient to use the bathroom), bathing, feeding, treating wounds or other concerns, assisting to stand, sit, or walk, transferring the patient (e.g., from a bed to a chair), and the like.

By automatically identifying these actions, the system is able to generate thorough and accurate records of the actions performed, which can have a large number of benefits. For example, because the records are more accurate and consistently-created, the system may be better able to identify trends in the patient's trajectory (e.g., an increasing frequency or length of various assistance needs, which may indicate a decline in the patient health). These trends are not apparent in conventional systems, as the manually-created records are generally high level and do not indicate the specific characteristics or context of the assistance. Further, because the charting can be performed automatically, the user has a reduced burden and is better able to assist residents or patients, rather than spending time recording what action(s) were performed.

### Example Environment for Training Machine Learning Models to Evaluate Sensor Data

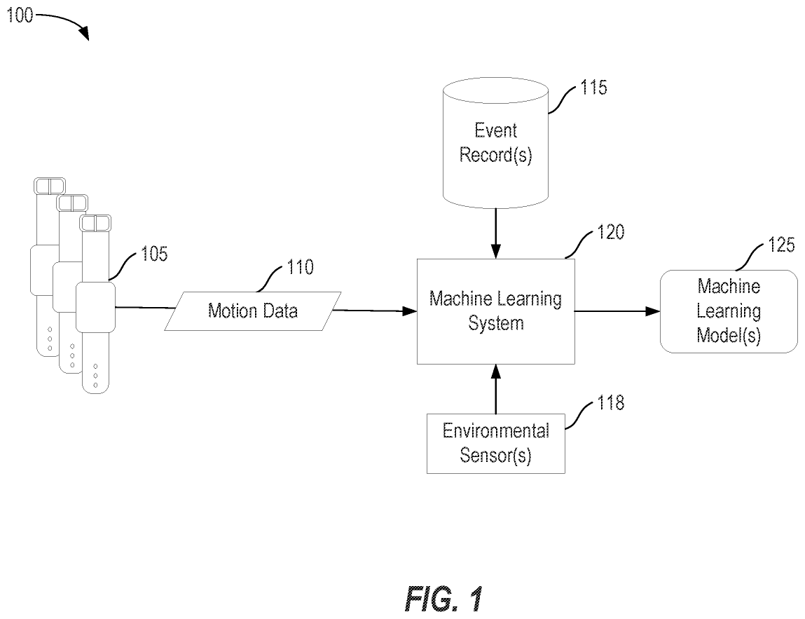

FIG. 1 depicts an example environment 100 for training machine learning models to evaluate sensor data and classify motion.

In the illustrated environment 100, a set of one or more motion sensors 105 are configured to record motion data from one or more users. In some embodiments, the motion sensors 105 are included in wearable devices. For example, the motion sensors may correspond to wrist-wearable devices such as smart watches. In an embodiment, the motion sensors 105 are generally configured to capture motion data of the user's arms, hands, and/or fingers. In embodiments, there may be any number of motion sensors 105 used in the environment 100. For example, the system may use two motion sensors 105 per user (e.g., one on each wrist), and there may be any number of users that are used to generate training data.

As illustrated, the motion sensors 105 transmit or otherwise provide motion data 110 to a machine learning system 120. The motion data 110 may generally be indicative of the orientation of one or more parts (e.g., the hands or arms) of a user at various points in time, movement of these parts (which may include the direction of movement and/or the acceleration or speed of the movement), and the like.

In various embodiments, this motion data 110 may be provided using any suitable technology, including wired or wireless communications. For example, in one embodiment, the motion sensors 105 can use cellular communication technology to transmit the motion data 110 to the machine learning system 120. In some embodiments, the motion sensors 105 use local wireless networks, such as a WiFi network, to transmit the motion data 110. In at least one embodiment, the motion sensors 105 can transmit the motion data 110 to one or more intermediary devices, which can then forward the data to the machine learning system 120. For example, the motion sensors 105 may transmit the motion data 110 to a smartphone, tablet, or other device associated with the user (e.g., via Bluetooth), and this user device can forward the data to the machine learning system 120 (e.g., via WiFi or a cellular connection).

In some embodiments, the motion data 110 is transmitted over one or more networks including the Internet. That is, the machine learning system 120 may reside at a location remote from the user and motion sensors 105 (e.g., in the cloud). Though a set of motion sensors 105 is illustrated for conceptual clarity, in embodiments, data from any number and variety of sensors may similarly be provided to the machine learning system 120. For example, as discussed above, the machine learning system 120 may receive data from proximity sensors, check-in devices, and the like.

In some embodiments, the motion data 110 is collected and/or transmitted continuously by the motion sensor(s) 105. That is, the motion sensors 105 may be continuously recording the movement of the user, and the machine learning system 120 can parse or delineate this data into defined segments, such as corresponding to windows of time, discrete actions being performed (as indicated by the event records 115), and the like. In some embodiments, the motion sensors 105 may alternatively or additionally transmit the motion data 110 in segments.

For example, the motion sensor 105 may only begin recording motion data 110 upon being triggered (e.g., by the user selecting a button or otherwise initiating recording). When the user has completed the action, they can similarly turn off the recording. In one such embodiment, the machine learning system 120 may evaluate only this window of relevant motion data 110 for the action. In a related embodiment, the motion sensor 105 may continuously record motion data 110, but only transmit it when the user indicates to do so. For example, upon completing an action, the user may cause the motion sensor 105 to transmit some portion of the previously-collected data to the machine learning system 120. This may include, for example, transmitting a defined window of data (e.g., the last ten minutes), transmitting a user-indicated window, and the like.

In the illustrated example, the environment 100 also includes one or more environmental sensors 118. For example, as discussed above, the environmental sensors may include sensors capable of generally sensing or detecting the presence and/or movement of users and patients, such as via cameras, thermal sensors, motion sensors, pressure sensors, and the like. In the illustrated embodiment, data from these environmental sensors 118 can also be provided to the machine learning system 120 to improve model training. For example, based on the determined movement(s) of the users or patients (e.g., moving towards the bathroom), the machine learning system 120 may be able to more accurately detect or predict actions being performed. In some embodiments, however, the environmental sensors 118 are not present (or not used), and the machine learning system 120 uses only motion data 110 from the motion sensors 105.

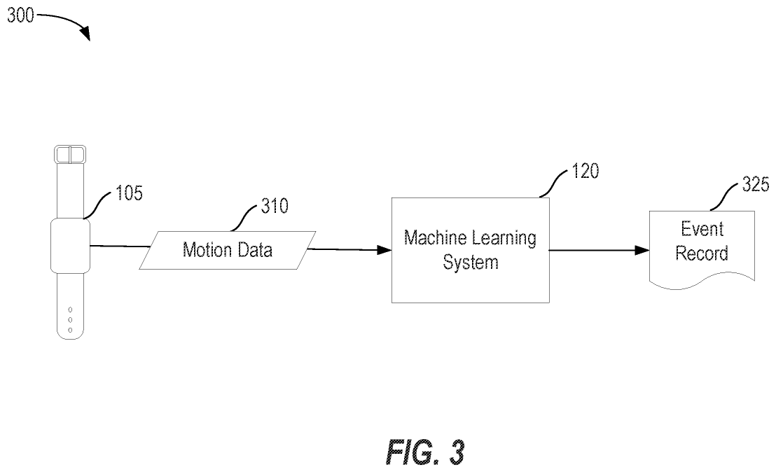

In embodiments, the machine learning system 120 can generally train one or more machine learning models 125 to analyze the motion data 110, and/or use trained models to evaluate the motion data 110 (discussed in more detail below with reference to FIG. 3). In the illustrated example, the machine learning system 120 can also receive a set of event records 115. In an embodiment, the event records 115 can generally include information relating to actions performed by one or more users, such as caregiving actions like bathing, feeding, transferring, and the like. For example, each event record 115 may indicate the user(s) who provided the assistance, the patient(s) that were assisted, the type of assistance (e.g., the specific actions performed), the location of the assistance, the time of the assistance, the duration of the assistance, and the like.

In the illustrated embodiment, the machine learning system 120 can use the event records 115 to automatically label some or all of the motion data 110. For example, when the machine learning system 120 receives motion data 110 for a given user, the machine learning system 120 may search the event records 115 to determine whether one or more event records 115 were created to reflect action(s) performed during that time. In one embodiment, if no such events are found, the machine learning system 120 may prompt the user to indicate what action(s) were performed.

In some embodiments, rather than attempting to identify an event record 115 that corresponds to motion data 110, the machine learning system 120 may identify motion data 110 that corresponds to a recorded event record 115. That is, the machine learning system 120 may parse the event records 115 to determine, for example, one or more action(s) performed by one or more user(s). For each such action, the machine learning system 120 can retrieve the received motion data 110 from the corresponding user at the corresponding time, and label this data as indicative that the user was performing the given action.

In one embodiment, to train the models, the machine learning system 120 can collect motion data 110 and event record(s) 115 over time from any number of users. For example, each user in a facility (e.g., a long term care unit) can have an associated set of one or more motion sensors 105, and the motion data 110 from each user can be used to train the models using a potentially tremendous amount of data, enabling far more accurate classifications.

Generally, as discussed in more detail below, training the machine learning models 125 includes providing some set of motion data 110 from one or more users as input to the model (e.g., motion data covering a defined window of time) to generate some predicted output (e.g., a predicted action that the user was performing when the motion data 110 was collected, or a probability or likelihood score for each of a set of possible actions). At early stages of training, this output may be relatively random or unreliable (e.g., due to the random weights and/or biases used to initiate the model). The predicted action (or probability scores) can then be compared against the known ground-truth label of the data (e.g., the actual action, as indicated in the event records 115) to generate a loss, and the loss can be used to refine the model (e.g., using back propagation in the case of a neural network).

Generally, this refinement process may be performed individually for each action or set of motion data (e.g., using stochastic gradient descent) or in batches (e.g., using batch gradient descent). Further, in embodiments, the machine learning system 120 can train models to operate on windows of data (e.g., a collection motion values during the window) or based on continuous data (e.g., a continuous stream of motion data).

In an embodiment, once the model(s) are trained, the machine learning system 120 can deploy them for use in real-time. In one embodiment, as discussed above, the models may be trained on one or more systems and deployed to one or more other systems. In other embodiments, the machine learning system 120 can both train the models and use them for inferencing.

### Example Workflow for Generating Labeled Data to Train Machine Learning Models

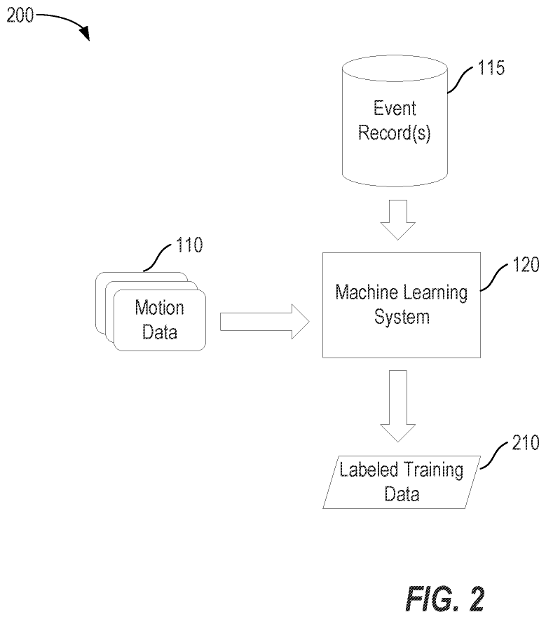

FIG. 2 depicts an example workflow 200 for generating labeled data to train machine learning models to classify motion.

In the illustrated workflow 200, the machine learning system 120 receives discrete records of motion data 110 and event records 115, and generates labeled training data 210. In an embodiment, as discussed above, the labeled training data 210 may generally correspond to some set of motion data 110 (e.g., from one or more wrist-mounted sensors of one or more users), where the label indicates the action(s) that the user was performing when the data was collected.

In some embodiments, the machine learning system 120 may parse the event records 115 to identify action(s) that were performed. For each such action, the machine learning system 120 can identify the user that performed the action (as indicated in the event record 115), as well as the time when the action was performed (or other contextual data that may be used to identify the corresponding motion data). Using this information, as discussed above, the machine learning system 120 can identify the relevant motion data 110.

In at least one embodiment, the machine learning system 120 labels the labeled training data 210 based not only on the action being performed, but also on the hand(s) being used to perform it. For example, the data may indicate whether the motion data corresponds to the user's left hand or right hand. As a large number of tasks and actions require two hands (often performing two different types of motion), this label may be useful in generating more accurate models. For example, the machine learning system 120 may train a first model to process data from the users' left hand, and a second model to process data from the right hand. This can result in improved predictions. In at least one embodiment, rather than labeling the data as left or right handed, the machine learning system 120 can label it as corresponding to the user's dominant hand or non-dominant hand, if applicable. In one such embodiment, the machine learning system 120 can train a first set of one or more models for right-hand dominant users, and a second set for left-hand dominant users.

In some embodiments, the machine learning system 120 generates labeled training data 210 that includes aggregated data from a number of users. That is, the machine learning system 120 can label the motion data 110 based on the underlying action(s), without reference to the particular user that performed the action. In such an embodiment, the labeled training data 210 can be used to train a single model, which may then be used to evaluate data from any given user. Such aggregated training may be particularly useful for actions where the particular motions used are generally consistent across populations.

In at least one embodiment, the machine learning system 120 can additionally or alternatively tag the labeled training data 210 to indicate which user(s) provided the data. Using this tagged data, the machine learning system 120 may train (or refine) separate models for each such user. That is, the machine learning system 120 may train personalized prediction models that are intended to process data from the corresponding user. For example, when motion data 110 is received during runtime, the machine learning system 120 may identify the corresponding model for that user, and process it accordingly. Such separate training may be particularly useful for actions where the particular motions differ according to personal preference.

In some embodiments, the machine learning system 120 can train a global model using labeled training data 210 from a set of users, and refine the model separately for one or more users using labeled training data 210 corresponding to those users. That is, for each relevant user, the machine learning system 120 may refine the global model using data for the relevant user in order to generate a personalized model for the user. This may enable the machine learning system 120 to retain the accuracy of the global model (achieved using large amounts of data from different users) while also enabling more personalized (and potentially more accurate) predictions.

In embodiments, the labeled training data 210 may be stored and used for future training or refinement, as appropriate. In some embodiments, the machine learning system 120 generates the labeled training data 210 continuously (e.g., as new data is received). For example, in one such embodiment, each time a new event record 115 is recorded, the machine learning system 120 can evaluate it to retrieve the corresponding motion data 110, and generate an exemplar of labeled training data 210. In a related embodiment, each time motion data 110 is received, the machine learning system 120 can determine the corresponding event record 115 (or, in some cases, prompt the user to indicate the action), and generate an exemplar of labeled training data 210. This can allow the machine learning system 120 to continuously update the set of labeled training data 210, such that it is ready for use at any time.

In some embodiments, the machine learning system 120 can alternatively update the labeled training data 210 periodically, or upon some defined criteria or occurrence. For example, the machine learning system 120 may periodically evaluate the event records 115 and/or motion data 110 (e.g., daily, weekly, and the like) to generate labeled training data 210. This may allow the machine learning system 120 to perform the label generating workflow 200 during defined times when the load on the system is low (such as overnight) which can reduce the computational burden on the machine learning system 120, thereby improving its ability to handle other workloads (such as the actual training or inferencing).

### Example Environment for Using Machine Learning to Classify Sensor Data

FIG. 3 depicts an example environment 300 for using machine learning models to classify sensor data. In one embodiment, in the environment 300, the machine learning system 120 uses a set of trained machine learning models (e.g., the machine learning models 125 of FIG. 1) to process and classify motion data during runtime.

The illustrated environment 300 corresponds to use of the models during an inferencing stage. As used herein, “inferencing” generally refers to the phase of machine learning where the model(s) have been trained, and are deployed to make predictions during runtime. As illustrated, during inferencing, the machine learning system 120 receives motion data 310 from one or more motion sensors 105.

In some embodiments, the machine learning system 120 receives this motion data 310 in real-time (or near real-time) from participating users. That is, the motion sensor 105 may transmit the motion data 310 continuously, as it is collected. In other embodiments, as discussed above, the motion sensors 105 transmit the data upon defined events, such as when the user instructs it to (e.g., via a button or verbal command).

The machine learning system 120 may generally process the motion data 310 using one or more trained machine learning models to identify and classify the action(s) that the user is performing. As discussed above, classifying the actions may include processing the data using multiple models. For example, the machine learning system 120 may use a first model to process data from the user's dominant side, and a second to process the data from their non-dominant side. This may allow the models to learn for the specific motion(s) used by each hand to perform the action(s), enabling more accurate action identification.

In at least one embodiment, the machine learning system 120 can determine whether the output of the dominant side model and non-dominant side model align. For example, the machine learning system 120 may determine whether each has classified the motion data 310 as the same action. If so, the machine learning system 120 may create an event record 325 indicating that the action was performed.

In one embodiment, if the models disagree as to what action was performed (or if the prediction is associated with a low confidence), the machine learning system 120 may take a variety of actions. In one such embodiment, the machine learning system 120 may evaluate the confidence of each prediction (from each model), and select the action having the highest confidence. In some embodiments, in the event of model disagreement, the machine learning system 120 can prompt the user to indicate what action was being performed. As discussed in more detail below, the machine learning system 120 may use the user's response to further train or refine the model(s).

In some embodiments, as discussed above, the machine learning system 120 may use a global model (shared across users) to classify the motion data 310. In other embodiments, the machine learning system 120 may use user-specific models (trained or refined based on data from a single user, or a set of users) to classify the data.

Though not included in the illustrated example, in some embodiments, one or more environmental sensors (such as environmental sensors 118 of FIG. 1) may also be used during the inferencing process. For example, the machine learning system 120 may train models to evaluate not only motion data, but also relevant environmental data from the same space, in order to determine or identify user actions.

In an embodiment, to generate the event record 325, the machine learning system 120 may additionally gather or determine other relevant information for the action. For example, based on the identity of the motion sensor 105 that provided the motion data 310, the machine learning system 120 can determine which user should be designated as the performer of the action.

Though not depicted in the illustrated example, in some embodiments, the machine learning system 120 can also consider other information, such as from one or more proximity sensors or check-in devices, in order to determine the relevant information for the event record 325. For example, based on a check-in scan and/or proximity data, the machine learning system 120 can determine which patient was being assisted, and whether one or more other user(s) also helped to provide assistance.

In some embodiments, if multiple patient(s) are indicated, the machine learning system 120 can take one or more actions to identify the specific patient (or patients, for some actions) that were assisted. For example, if two or more patients share a room, scanning into the room may not uniquely indicate which patient is being assisted. Similarly, if assistance is being offered in a public area, proximity data may indicate that multiple patients are nearby, and thus it is difficult to determine which patient was specifically being assisted.

In some embodiments, the machine learning system 120 can infer which patient was being assisted based on relevant patient records or characteristics. In one such embodiment, for each individual that was potentially being assisted, the machine learning system 120 may retrieve a set of patient records indicating assistance needs, and identify the correct patient(s) based on these records. For example, suppose the machine learning model(s) indicate that the user was feeding a patient. If only one of the nearby or relevant patient(s) needs assistance with eating, the machine learning system 120 can determine or infer that this patient should be included in the event record 325, rather than the other patient(s). In at least one embodiment, the machine learning system 120 can additionally or alternatively identify the specific patient(s) by prompting the user to indicate which patient they were assisting.

In one embodiment, if multiple user(s) may be involved in the assistance, the machine learning system 120 can take various actions to determine which user(s) assisted, as well as which user should be the primary user for the event record 325. For example, if multiple users are reflected in the check-in data and/or proximity data, the machine learning system 120 can evaluate motion data from each such user to determine whether they were performing an action or assisting in one (as opposed to observing, or simply standing nearby). In at least one embodiment, of the participating users, the machine learning system 120 can identify a primary user based on a variety of criteria, including seniority, which specific portion(s) of the action each user is performing, and the like. By identifying a primary user, the machine learning system 120 can prevent multiple (duplicative) event records 325 from being created.

In at least one embodiment, prior to finalizing the event record 325, the machine learning system 120 can first verify its contents based on a variety of criteria. For example, the machine learning system 120 may confirm that the predicted action (indicated by the machine learning model(s)) is associated with a confidence value that meets a defined threshold. That is, if the machine learning model outputs a confidence in its classification, the machine learning system 120 can confirm that the confidence exceeds a minimum value. If not, the machine learning system 120 may flag the event record 325 as unverified (or refrain from creating it entirely).

In one embodiments, the machine learning system 120 can verify the record by evaluating various patient data. In one such embodiment, as discussed above, the machine learning system 120 may confirm that the determined patient actually needs the determined assistance. For example, if the patient records indicate that they do not need assistance eating, but the model(s) classified the motion data 310 as assisting a patient to eat, the machine learning system 120 may flag the event record 325 as unverified (or refrain from creating it).

In some embodiments, the machine learning system 120 can cause some or all of the event records 325 to be manually reviewed and verified, prior to finalizing them. For example, the machine learning system 120 may identify the primary user, and transmit the event record 325 to this user for approval. In some aspects, this approval process is used for all event records 325. In one embodiment, the machine learning system 120 only seeks approval during an initial testing stage of deployment (e.g., after the models are trained but before they have been in use and confirmed to be accurate). Once the model(s) are confirmed to be mature, in such an embodiment, the machine learning system 120 can cease the approval process.

In some embodiments, the machine learning system 120 may randomly select a subset of the event records 325 for approval, in order to confirm that the model(s) are functioning accurately. Further, in some embodiments, the machine learning system 120 may prompt for approval only when various conditions are met (such as minimum confidence, conflicting patient data, and the like), as discussed above.

Though some embodiments may therefore still involve manual approval, the machine learning system 120 is nevertheless able to significantly reduce manual effort in the charting process. For example, because the machine learning system 120 can automatically generate all or most of the event record 325, the user need not enter it. Even if some data cannot be determined (e.g., if the machine learning system 120 cannot determine which specific patient was being assisted), the event record 325 may nevertheless indicate some relatively small set of alternative patients, allowing the user to quickly select the correct one.

Though the illustrated example depicts the machine learning system 120 performing inferencing, in some embodiments, as discussed above, training and inferencing may be performed on separate systems. For example, a centralized system may train the models (e.g., using data from multiple users and/or multiple facilities), and the models may be distributed to each local facility for inferencing by local system(s).

### Example Method for Training Machine Learning Models to Classify Motion Data

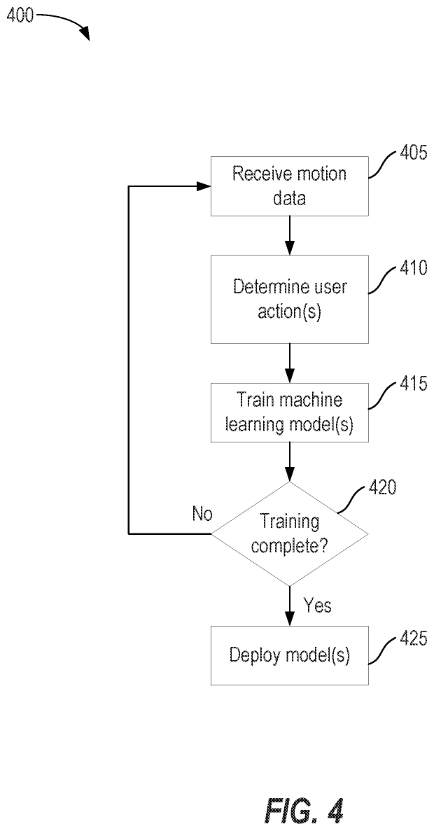

FIG. 4 is a flow diagram depicting an example method 400 for training machine learning models to classify motion data. In some embodiments, the method 400 is performed by a machine learning system, such as machine learning system 120 of FIGS. 1 and 2.

At block 405, the machine learning system receives motion data. As discussed above, this motion data may generally be indicative of the orientation and/or movement of one or more parts of a user, such as a caregiver, nurse, and the like. In some embodiments, the motion data is collected via one or more wearable sensors of the user. In at least one embodiment, the motion data includes a first set of data collected from one hand or arm (e.g., using a wrist-mounted sensor on the user's dominant side) and a second set of data collected from the other hand or arm (e.g., via a wrist-mounted sensor on the user's non-dominant side).

In some embodiments, the motion data corresponds to accelerometer data. For example, each motion sensor may include one or more accelerometers that can be used to determine orientation of the device (and, therefore, the user's arm or hand) and/or movement. In at least one embodiment, each motion sensor device includes a three-axis accelerometer (e.g., an accelerometer that can measure acceleration in each of three dimensions). This can enable the device to determine its orientation and movement in a three-dimensional space.

In some embodiments, the motion data contains relatively raw data, such as the detected acceleration along each axis at each point in time. In other embodiments, the motion sensor device can perform some preprocessing on the data, and transmit more detailed information, such as the determined device orientation and movement.

At block 410, the machine learning system can determine one or more user action(s) that correspond to the motion data. For example, in one embodiment, the machine learning system determines the time of that the motion data was captured, as well as the user that performed the motion. The machine learning system can then retrieve one or more records (e.g., event records, as discussed above) for the corresponding user and time. This can allow the machine learning system to determine what action(s) were being performed when the motion data was captured.

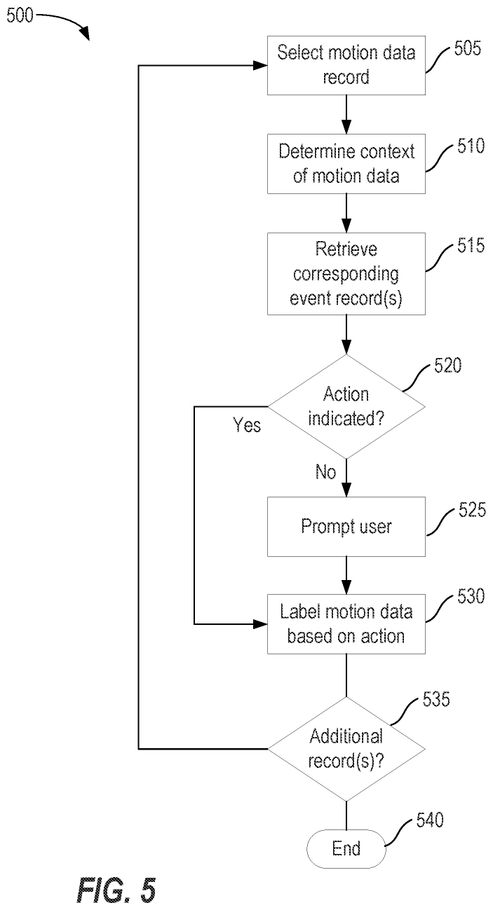

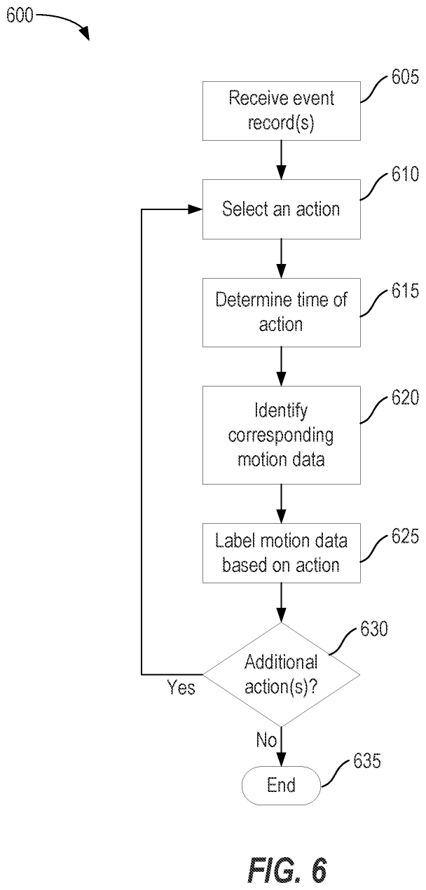

In some embodiments, the machine learning system may instead evaluate the event records to identify actions. For each such action, the machine learning system can determine the time of the action and the user that performed it, and then identify the relevant motion data that corresponds to this user and time. Some examples of determining the user action(s) that correspond to motion data are described below in more detail with reference to FIGS. 5 and 6.

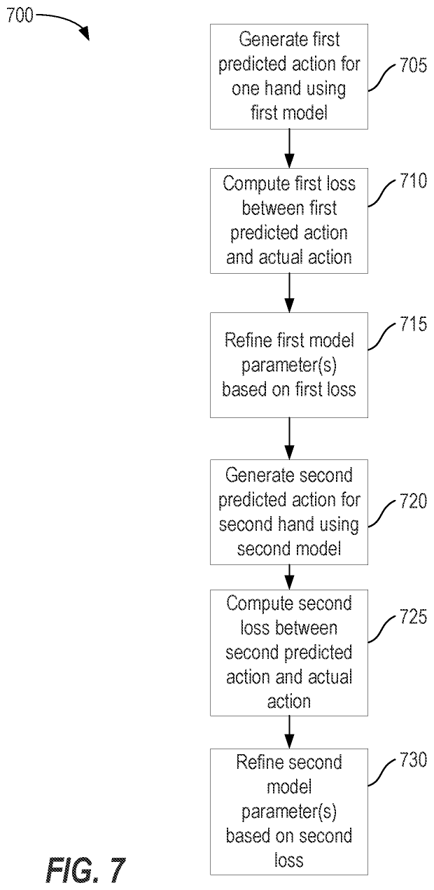

At block 415, once the motion data and corresponding action (or the action and corresponding motion data) have been determined, the machine learning system trains one or more machine learning models based on the labeled data. One example technique for training the machine learning model(s) is described in more detail below with reference to FIG. 7.

In some embodiments, the machine learning system trains a single model to classify the motion data. That is, a single model may be trained to receive and classify motion data from multiple sensors (e.g., from both the left and right hands of the user), in order to predict what action the user is performing (using one or both hands). In other embodiments, the machine learning system may train multiple models (e.g., one for each sensor or hand).

In one embodiment, the machine learning system trains a global model to classify motion data from any user. In some embodiments, as discussed above, the machine learning system may train (or refine) separate models for each user. For example, at block 415, the machine learning system may select the model that corresponds to the specific user that provided the motion data, and refine this model.

Generally, during the training process, the model(s) can be iteratively refined to produce more accurate motion classifications. In some embodiments, the models may be initialized using random parameters, resulting in effectively random classifications. However, as these parameters are refined over time using labeled data (as discussed below in more detail), they can generally learn to generate accurate classifications for the motion data.

Although the illustrated method 400 depicts training the model individually for each action, in some embodiments, the machine learning system may train the model for batches of data. For example, using stochastic gradient descent, the machine learning system may compute a loss based on a single action and motion data pair, and refine the model using this loss. Using batch gradient descent, the machine learning system may compute a loss based on a batch of actions and corresponding labels, refining the model based on this batch of data.

At block 420, the machine learning system determines whether training is complete. In embodiments, this can include evaluation of a wide variety of termination criteria. For example, in one embodiment, the machine learning system can determine whether training is complete based on whether there is any additional training data available. If at least one exemplar remains, the method 400 may return to block 405. In some embodiments, the machine learning system can additionally or alternatively determine whether a maximum amount of time, a maximum number of training cycles or epochs, and/or a maximum amount of computing resources have been spent training the model(s). If not, the method 400 returns to block 405.

In at least one embodiment, to determine whether training is complete, the machine learning system can determine whether the model(s) have reached some preferred minimum accuracy. For example, using a set of testing data (e.g., motion data, labeled with the corresponding action, that has not been used to train the model), the machine learning system can test the accuracy of the predictions (such as by processing the motion data using the model, and determining how often the classified output matches the actual label). If the model accuracy is still not satisfactory, the method 400 returns to block 405.

Regardless of the specific termination criteria used, once the machine learning system determines that training is complete, the method 400 continues to block 425, where the machine learning system deploys the trained model(s) for runtime. In some embodiments, as discussed above, the machine learning system can use the model(s) locally to perform inferencing. That is, the machine learning system may be used to both train the models, as well as use them to classify motion data during runtime. In some embodiments, the machine learning system may additionally or alternatively provide the models to one or more other systems that perform runtime inferencing.

### Example Method for Generating Labeled Data to Train Machine Learning Models

FIG. 5 is a flow diagram depicting an example method 500 for generating labeled data to train machine learning models to classify motions. In some embodiments, the method 500 is performed by a machine learning system, such as machine learning system 120 of FIGS. 1 and 2. In one embodiment, the method 500 provides additional detail for blocks 405 and/or 410 of FIG. 4, where the machine learning system correlates motion data and event data.

At block 505, the machine learning system selects a record of motion data. For example, as discussed above, the motion data may be delineated and/or stored into windows of time. These delineations may be fixed (e.g., into fixed thirty second windows) or dynamic (e.g., where the length of the window may vary, depending at least in part on the action being performed). For example, in some embodiments, at least during a training stage, the user may manually indicate when they begin and end a given action, allowing the machine learning system to readily delineate the data into records. In some embodiments, rather than evaluate the motion data in discrete windows, the machine learning system may be configured to evaluate and classify continuous streams of data for classification.

In an embodiment, the machine learning system may use any suitable criteria or technique to select the motion data record at block 505, as all relevant motion data will be similarly evaluated during the labeling process. Though the illustrated example depicts a sequential process (iteratively evaluating each motion data record in turn) for conceptual clarity, in some embodiments, the machine learning system can evaluate some or all of the motion data entirely or partially in parallel.

At block 510, the machine learning system determines the context of the selected motion data record. In embodiments, the context may include a variety of detail, such as the time at which the motion data was collected, the date when the data was collected, the user associated with the data (e.g., the user that was wearing the motion sensor(s)), and the like. For example, to determine the time of the data, the machine learning system may determine the timestamp of the beginning of the motion data (e.g., at the start of the window), the timestamp at the end of the data (e.g., at the end of the window), and the like.

At block 515, based on this contextual data, the machine learning system can retrieve a corresponding set of one or more event records, if they exist. For example, the machine learning system may identify the user indicated in the motion data record (e.g., the user who was wearing the motion sensor(s)), and retrieve event records that indicate this user (either as the primary user, or as an assisting user). The machine learning system can then identify the record(s) that indicate or are associated with a time corresponding to the time of the motion data.

For example, in one embodiment, the machine learning system can identify any event record(s) indicating that the user performed an action at a time that matches the timestamp(s) of the motion data, or is within a defined threshold (e.g., within fifteen minutes). In some embodiments, the machine learning system can identify any records that were recorded on the same day or during the same shift as the motion data (e.g., if the user charts their actions periodically, rather than immediately after performing an action). In one such embodiment, the machine learning system can identify the relevant record(s) from among these identified records based on further contextual data, such as the identity of the patient indicated in the record (which should match the patient associated with the motion data, as determined using check-in scans or proximity data, for example).

Once the corresponding event record(s) for the motion data have been identified, the method 500 continues to block 520, where the machine learning system determines whether the record(s) indicate an action that was performed by the user at the time. In at least one embodiment, if no corresponding event record(s) can be found (or if there are more than one possible alternatives, and the machine learning system cannot determine which event record corresponds to the motion), the method 500 can also continue to block 520, where the machine learning system will determine that no action(s) are indicated.

If, at block 520, the machine learning system determines that the relevant event record(s) do identify an action performed by the user, the method 500 can bypass block 525 and proceed to block 530, discussed in more detail below. If the machine learning system determines that no action is indicated (e.g., there is no corresponding event record, the event record does not indicate what action was taken, and/or there are multiple alternative action(s) or event record(s) that might correspond to the motion), the method 500 continues to block 525.

At block 525, the machine learning system prompts the relevant user(s) to indicate what action was taken. For example, the machine learning system may transmit a notification or alert to the user (e.g., via an email or text message) indicating the context of the motion data (e.g., when it was recorded, where in the facility the user was at the time of recording, what patient was being assisted, and the like). In some embodiments, if the machine learning system was able to identify a subset of possible actions (e.g., from two or more event records), the machine learning system can suggest this identified subset. The user can then respond to this prompt by indicating what action they performed, if they recall. In some embodiments, if the user cannot recall what action was performed (or does not respond), the motion data may be discarded, and the machine learning system will refrain from training the model based on that data.

At block 530, the machine learning system labels the selected motion data with the determined or indicated action that was performed. As discussed above, the machine learning system can then use this labeled data to train one or more machine learning models to classify motion data based on the action being performed.

At block 535, the machine learning system determines whether there are any additional motion data record(s) that have not yet been evaluated. If so, the method 500 returns to block 505. If not, the method 500 terminates at block 540.

### Example Method for Generating Labeled Data to Train Machine Learning Models

FIG. 6 is a flow diagram depicting an example method 600 for generating labeled data to train machine learning models to classify motions. In some embodiments, the method 600 is performed by a machine learning system, such as machine learning system 120 of FIGS. 1 and 2. In one embodiment, the method 600 provides additional detail for blocks 405 and/or 410 of FIG. 4, where the machine learning system correlates motion data and event data. In one embodiment, the method 600 presents an alternative approach to labeling data, as compared to the method 500 in FIG. 5. Specifically, the method 500 begins with motion data and attempts to identify corresponding event records, while the method 600 begins with event records and attempts to identify corresponding motion data.

At block 605, the machine learning system receives one or more event records. As discussed above, these event records can generally correspond to charts or reports prepared by users (e.g., caregivers) indicating actions performed to assist residents or patients. For example, after helping a resident bathe, the user may prepare a corresponding event record indicating the action, as well as relevant contextual information (such as the identity of the patient, the time of beginning and/or finishing the assistance, the location of the assistance, any notes they have, such as the resident's mood or requests, and the like). In one embodiment, each event record specifies zero or more assistance actions. For example, in one such embodiment, a user may create an event record indicating something the patient did or said, even if the user did not perform any assistance actions. Similarly, in some embodiments, the user may create a single record indicating multiple actions (e.g., “assisted resident with bath, fed dinner, and helped into bed.”

At block 610, the machine learning system selects an action indicated in the event records. Generally, the machine learning system may use any suitable technique to select the action, as all actions will be evaluated in turn to generate labeled data. For example, the machine learning system may select the earliest-performed action that has not yet been used to train or refine the model(s). Although the illustrated example depicts sequential processing of the actions/event records for conceptual clarity (e.g., processing one action at a time), in some embodiments, the machine learning system can process some or all in parallel.

At block 615, the machine learning system determines the time when the selected action was performed. For example, the corresponding event record (that indicates the action) may note the time the action began, the time it ended, and the like. In some embodiments, the record indicates a relatively precise time. In others, it may indicate more broad or generic times (e.g., “around noon,” “early afternoon,” or “today”). In some embodiments, to assist in detecting the relevant motion data, the machine learning system can also determine other contextual information, such as the location of the assistance, the patient that was assisted, and the like.

At block 620, the machine learning system identifies the motion data that corresponds to the selected event. For example, based on the identity of the user that performed the action and the determined time of the action (and, in some embodiments, other context such as the resident being assisted), the machine learning system can identify a window of motion data that was recorded when the action was being performed.

In at least one embodiment, as discussed above, if the machine learning system cannot conclusively identify the motion data, the machine learning system may prompt the user to provide more information. If the user cannot do so (e.g., cannot be more specific about when the action was performed), as discussed above, the machine learning system may refrain from using the selected action to train the model.

At block 625, the machine learning system then labels the identified motion data based on the selected action. This labeled data can then be used to train or refine one or more machine learning models to classify input motion based on the action being performed, as discussed above.

At block 630, the machine learning system determines whether there is at least one additional action reflected in the event record(s) that has not yet been evaluated. If so, the method 600 returns to block 610. If not, the method 600 terminates at block 635.

### Example Method for Refining Machine Learning Models to Predict Actions

FIG. 7 is a flow diagram depicting an example method 700 for refining machine learning models to predict actions. In some embodiments, the method 700 is performed by a machine learning system, such as machine learning system 120 of FIGS. 1 and 2. In one embodiment, the method 700 provides additional detail for block 415 of FIG. 4, where the machine learning system trains the machine learning model(s).

At block 705, the machine learning system generates a first predicted action for a first hand of a user by processing motion data from that hand using a first machine learning model. For example, the first machine learning model may be a model trained to process data from a user's left hand and/or from their non-dominant hand. In at least one embodiment, the machine learning system trains multiple sets of models including, for example, a first set (including a first left hand model and a first right hand model) for left-handed users, a second set (including a second left hand model and a second right hand model) for right-handed users, and/or a third set (including a third left hand model and a third right hand model) for ambidextrous users.

At block 710, the machine learning system computes a first loss between the first predicted action (generated at block 705) and an actual ground-truth action for the motion data that was used to generate the predicted action (e.g., indicated in a label). In at least one embodiment, the actual model output (or the output at a penultimate layer of the model) is a set of scores, one for each class (e.g., for each possible action), and the predicted action is selected by identifying the output with the highest score. In one such embodiment, to generate the loss, the machine learning system can use various loss algorithms that seek to maximize the score of the “correct” class while minimizing the scores of the “incorrect” classes, such as hinge loss, cross entropy loss, and the like.

At block 715, the machine learning system can then refine the parameters (e.g., weights and/or biases) of the first model based on the loss generated in block 710. In this way, after the refinement, the first model would generate a somewhat different output (e.g., a different predicted action, or different score(s) for the possible actions) if it processed the same input. After a number of training rounds and/or epochs, the model learns to accurately classify the input data.

At block 720, the machine learning system can generate a second predicted action for the second hand of the user, by processing motion data from that second hand using a second machine learning model. For example, as discussed above, this second model may be trained for right-hand data from right-handed users.

At block 725, in a similar manner to the above discussion, the machine learning system can compute a second loss between the second predicted action or action scores (generated at block 720) and the actual ground-truth action for the motion data that was used to generate the predicted action (e.g., indicated in a label).

At block 730, the machine learning system can then refine the parameters (e.g., weights and/or biases) of the second model based on the second loss. As above, this causes the second model to similarly generate more accurate predictions or classifications of input data.

### Example Method for Using Machine Learning to Evaluate Sensor Data and Predict Actions

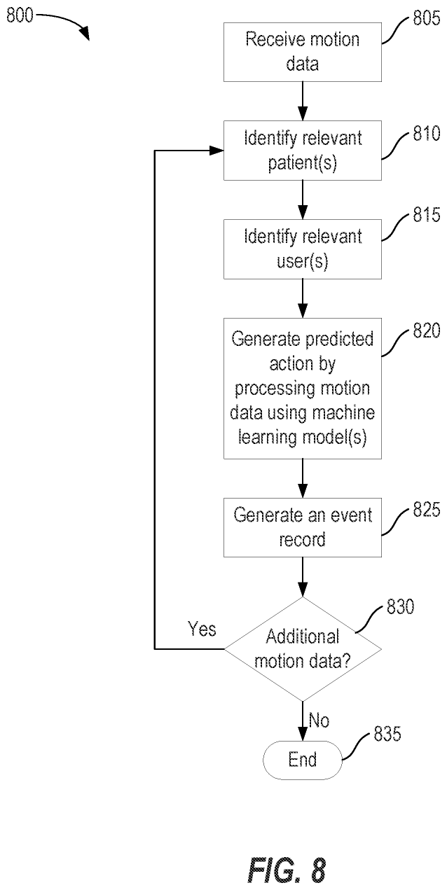

FIG. 8 is a flow diagram depicting an example method 800 for using trained machine learning models to evaluate sensor data and predict actions. In some embodiments, the method 800 is performed by a machine learning system, such as machine learning system 120 of FIGS. 1 and 2.

At block 805, the machine learning system receives motion data from a user. In an embodiment, this motion data is generally captured by one or more motion sensors (such as wrist-mounted wearables) and is transmitted to the machine learning system for machine learning-based classification, in order to enable automated recordation of the performed action(s). In at least one embodiment, as discussed above, the motion data corresponds to accelerometer data indicating orientation and/or movement of one or both hands or arms of a user.

In embodiments, as discussed above, the motion data may be transmitted from the motion sensor(s) directly to the machine learning system via one or more wireless networks, or may be transmitted through one or more intermediate devices. For example, the motion sensor may transmit the data to a user device such as a smartphone of the user, which can then forward it to the machine learning system.

In some embodiments, as discussed above, the motion data may be continuously transmitted to the machine learning system, or may be transmitted during windows and/or in batches. For example, the motion sensors may only transmit the motion data when instructed to do so by a user. Similarly, the motion data may be transmitted as it is collected, or may be transmitted as a block of data after the action is completed.

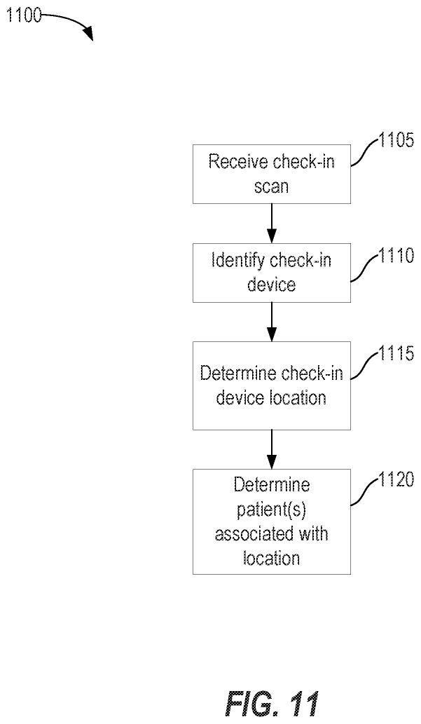

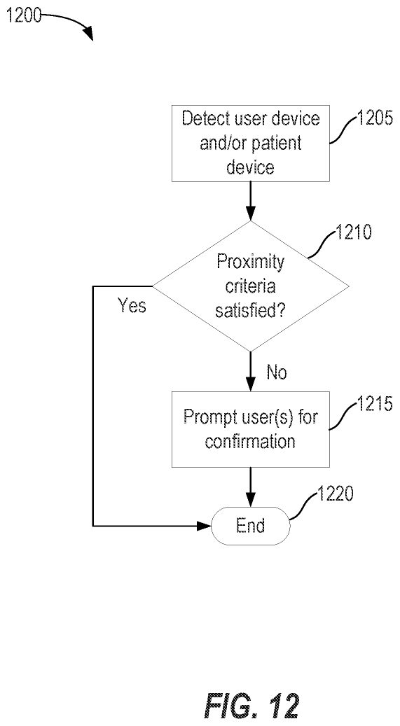

At block 810, the machine learning system identifies one or more relevant patients for the motion data. That is, the machine learning system can identify the patient(s) who were being assisted when the motion data was recorded. In some embodiments, the machine learning system does so using a check-in scanning system, as discussed above and in more detail below with reference to FIG. 11. In some embodiments, the machine learning system identifies the patient(s) using one or more proximity-based sensors, as discussed above and in more detail below with reference to FIG. 12.

In at least one embodiment, the machine learning system identifies the patient(s) based on input from the user. For example, the user may indicate (e.g., via text or verbally) the patient(s) that are being assisted (or were assisted). In one such embodiment, the machine learning system can use this indication as the patient identifier.

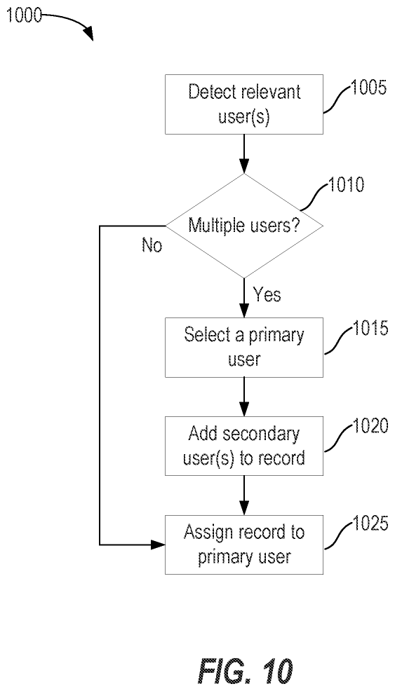

At block 815, the machine learning system identifies one or more relevant user(s) for the motion data. For example, the machine learning system may identify the user that the motion data corresponds to (e.g., because the user was wearing the motion sensors). In some embodiments, the machine learning system may additionally or alternatively use other means to identify the user(s), such as via check-in scans and/or proximity sensors. For example, the machine learning system may determine whether any additional users assisted in the action by evaluating the check-in scans, the users detected via proximity sensor(s) during the action, and the like. One example technique for identifying the user(s) is discussed in more detail below with reference to FIG. 10.

In at least one embodiment, the machine learning system can additionally or alternatively identify the user(s) based on input from the user(s). For example, the user(s) may indicate (e.g., via text or verbally) that they provided the assistance. In one such embodiment, the machine learning system can use this indication as the user identifier.

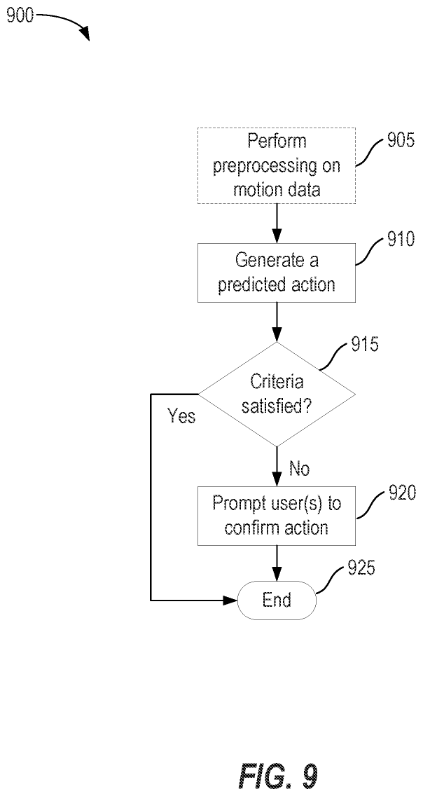

At block 820, the machine learning system generates a predicted action by processing the motion data using one or more machine learning models. One example technique for generating the predicted action is discussed in more detail below, with reference to FIG. 9. In some embodiments, as discussed above, the machine learning system may use a different set of one or more models, depending on the particular user reflected by the motion data.

For example, in one embodiment, the machine learning system uses user-specific models that were trained or fine-tuned specifically for the individual user. In some embodiments, as discussed above, the machine learning system may use one set of models for left-handed users, and a second set for right-handed users. In other embodiments, the machine learning system uses the same model(s) for all users.

At block 825, the machine learning system generates an event record reflecting the predicted action, relevant user(s), and relevant patient(s). In at least one embodiment, prior to, during, or after generating the event record, the machine learning system can verify or validate the information in it. For example, if the patient was determined from a user-provided indication, the machine learning system may determine whether the user-indicated patient matches the inferred or determined patient for the action. Similarly, if the participating user(s) were indicated manually by one or more users, the machine learning system can determine whether the indicated user(s) match the inferred or determined user(s) for the action. In the event of a mismatch, the machine learning system may prompt the user for confirmation, or take other actions.

In some embodiments, the machine learning system can evaluate the predicted action in view of one or more records of the relevant patient(s). In one such embodiment, the machine learning system may determine whether the patient records indicate that the relevant patient needs or receives the type of assistance that was predicted. For example, if the action includes helping a patient use the toilet, the machine learning system can determine whether the patient's chart indicates that they need assistance with this action, or if they have sufficient mobility.

In an embodiment, if the machine learning system fails to validate or verify the record, it can be provided to a user (e.g., the user reflected by the motion data) for verification or correction. In some embodiments, as discussed above, the machine learning system may fail to verify the record because some aspects of it conflict with other data (e.g., because the indicated user was not present in the facility that day, because the patient does not need the type of assistance indicated, and the like). In at least one embodiment, as discussed above, the machine learning system may also fail to verify a record if it is incomplete (e.g., because the machine learning system could not determine the identity of the patient with sufficient confidence).

At block 830, the machine learning system determines whether there is any additional motion data that has not yet been evaluated. For example, the received motion data (at block 805) may include multiple blocks or segments of data (e.g., corresponding to multiple actions performed to assist the same patient, or to multiple actions for multiple patients).

If no additional data remains, the method 800 terminates at block 835. If the motion data includes at least some set of data that has not yet been evaluated, the method 800 returns to block 810.

### Example Method for Evaluating Motion Data Using Machine Learning Models

FIG. 9 is a flow diagram depicting an example method 900 for evaluating motion data using machine learning models. In some embodiments, the method 900 is performed by a machine learning system, such as machine learning system 120 of FIGS. 1 and 2. In one embodiment, the method 900 provides additional detail for block 820 of FIG. 8, where the machine learning system generates a predicted action using machine learning model(s).

At block 905, the machine learning system can optionally perform any needed preprocessing on received motion data. For example, as discussed above, the machine learning system may delineate the data into windows or segments, which may include dividing it into defined segments to cover fixed lengths of time, or into dynamic segments based on the data itself. In at least one embodiment, the machine learning system can determine or infer breaks or pauses between actions (e.g., based on the magnitude or frequency of motions), and delineate the motion data into segments based on these pauses.

Generally, the preprocessing performed in block 905 can include a wide variety of operations depending on the particular implementation. For example, the machine learning system may apply one or more smoothing algorithms to the data, remove outliers, convert it to another coordinate frame, and the like.

At block 910, the machine learning system generates a predicted action using the data. In embodiments, the machine learning system may generate the predicted action by using one or more trained classifier models to classify the input motion data based on the action that the user was performing when the motion data was captured. As discussed above, these models may have been trained (by the machine learning system or by some other system(s) or component(s)) based on labeled motion data to generally identify and classify a variety of user actions, such as assistive or caregiving actions, depending on the particular implementation.

In some embodiments, the machine learning system uses a set of multiple machine learning models to classify the motion data. This may include selecting model(s) from among a set of models based on the context of the data, and/or processing it using multiple models. For example, in some embodiments, the machine learning system can use a model that was specifically trained or fine-tuned for the particular user or facility, as discussed above. Similarly, the machine learning system may use different models depending on the dominant hand of the user.

In at least one embodiment, the machine learning system can process different portions of the motion data using different models. For example, as discussed above, the machine learning system may process motion data from the user's left hand using a first model, and process motion data from the user's right hand using a second model.

At block 915, the machine learning system determines whether the predicted action satisfies one or more defined criteria. In embodiments, this criteria may vary depending on the particular implementation. In some embodiments, the criteria includes evaluating of the confidence of the prediction. For example, if the model outputs a classification confidence, the machine learning system may determine whether this confidence meets or exceeds a defined threshold. In at least one embodiment, as discussed above, the model may be configured to output a probability or likelihood score for each possible action, and the predicted action may correspond to the highest-scored action. In some embodiments, at block 915, the machine learning system can determine whether the score exceeds a threshold (e.g., greater than 50%).

In some embodiments, at block 915, the machine learning system can determine whether the model outputs (e.g., if two or more models were used) match or align. For example, if the machine learning system uses one model for data from the user's left hand and a second for data from the user's right, the machine learning system can determine whether these models agree on the predicted action.

If, at block 915, the machine learning system determines that the criteria are satisfied, the method 900 terminates at block 925. If the machine learning system determines that one or more of the criteria are not satisfied, the method 900 continues to block 920, where the machine learning system prompts the user to confirm the action that was performed. Though this may require some manual effort from the user, it can nevertheless generally reduce the amount of time and effort required to ensure accurate and complete records are generated.

In some embodiments, the machine learning system may indicate the predicted action(s) and/or one or more of the next-highest scored alternatives, and ask the user to confirm which is correct (or to indicate the action, if the suggestion list does not include the actual action). As the user need only select from a shortened list of alternatives, they can generally do so more quickly and accurately than if they created the entire record. Further, in embodiments, the other field(s) of the record may still be automatically populated, as discussed above. After the user confirms or corrects the action, the method 900 terminates at block 925.

### Example Method for Identifying Relevant User(s) for Predicted Actions

FIG. 10 is a flow diagram depicting an example method for identifying relevant user(s) for predicted actions. In some embodiments, the method 1000 is performed by a machine learning system, such as machine learning system 120 of FIGS. 1 and 2. In one embodiment, the method 1000 provides additional detail for block 815 of FIG. 8, where the machine learning system identifies the relevant user(s) for an action.

At block 1005, the machine learning system detects a set of user(s) that are relevant for a given action. The specific technique(s) used to detect the relevant users may vary depending on the particular implementation. In some embodiments, as discussed above, users may perform a check-in scan (e.g., by swiping their badge on a device near the entry to the patient's room) prior to entering and rendering assistance. In one such embodiment, the machine learning system can use this check-in data to identify the user(s) associated with the action.

In some embodiments, the machine learning system can use proximity data to identify the user(s). In one such embodiment, as discussed above, the users and/or patients may carry, wear, or otherwise be associated with proximity device(s) configured to detect the presence of other proximity device(s). For example, via Bluetooth, NFC, or other wireless communications, the user device(s) and patient device(s) may identify each other. In one embodiment, detecting the relevant user(s) includes identifying all user(s) (e.g., all user device(s)) that are detected during the action, within a defined distance during the action, and/or have a signal strength exceeding a defined threshold during the action (indicating proximity).

At block 1010, the machine learning system determines whether multiple users were identified for the action. If not, the method 1000 continues to block 1025, where the machine learning system assigns the (only) identified user as the primary user on the generated event record. That is, if no other user(s) scanned into the room and/or were detected in proximity to the assistance event, the machine learning system can determine that the single user (e.g., the user reflected by the motion data) was the only relevant user for the action.

Returning to block 1010, if the machine learning system determines that there were multiple users participating in the assistance, the method 1000 continues to block 1015, where the machine learning system selects a primary user for the action. In embodiments, the machine learning system can use a wide variety of criteria to select the primary user. For example, in one embodiment, the machine learning system determines the seniority or priority of the relevant user(s), and selects the most or least senior (or the user with the highest or lowest priority) as the primary user.

In some embodiments, the machine learning system selects the primary user based on the action(s) performed by each participating user. For example, the machine learning system may receive and process motion data from each relevant user (e.g., using the machine learning model) to determine what portion(s) of the action each user performed. The machine learning system can then identify the primary user as the one who performed a defined portion of the action (e.g., the most complex or important part, as defined in one or more rules defining how actions apportioned among multiple users).

At block 1020, the machine learning system adds the other (non-primary) users as assisting users to the event record, and at block 1025, the selected primary user is assigned to the record. In embodiments, by identifying the primary user, the machine learning system can prevent multiple entries from being created for the same action. Further, in the event that manual review is needed, the machine learning system can enable the primary user to perform this review, rather than prompting all of the involved users.

### Example Method for Identifying Relevant Patient(s) for Predicted Actions

FIG. 11 is a flow diagram depicting an example method 1100 for identifying relevant patient(s) for predicted actions. In some embodiments, the method 1100 is performed by a machine learning system, such as machine learning system 120 of FIGS. 1 and 2. In one embodiment, the method 1100 provides additional detail for block 810 of FIG. 8, where the machine learning system identifies the relevant patient(s) for an action.

At block 1105, the machine learning system receives a check-in scan for a user device. As discussed above, in some embodiments, the user(s) may use a device to check-in as they enter a room and/or begin assistance for the patient(s). In embodiments, the particular technology used to perform the check-in may vary.

For example, in some embodiments, the check-in device is a scanner or reader in the facility, such as an RFID reader or NFC scanner near the door of a room. In such an embodiment, the user device may be an RFID tag and/or NFC-capable device that is read, by the check-in device, when the user touches it to the reader (or places it in proximity to the reader). In some embodiments, the user device can additionally or alternatively be active. For example, the user's device may be used as the check-in reader (e.g., the RFID or NFC scanner), and the user can use it to scan or read an RFID tag or NFC chip in the facility (e.g., on or near the door to the room).

In at least one embodiment, the machine learning system uses visual check-in data. For example, the user may scan their ID badge (e.g., a barcode or QR code on the badge) using a visual scanner in the facility. Alternatively, the user may use a visual scanner to scan a barcode or QR code affixed to the door, or to the adjacent wall.

Regardless of the particular methodology used to generate the scan, these check-ins can be used to monitor the user's movements and actions throughout a facility, and can generally be used to indicate what user(s) participated in a given action or assistance event.

At block 1110, the machine learning system identifies the check-in device for the scan. For example, if the check-in system uses RFID or NFC readers in the facility, the machine learning system can identify the specific reader that received the check-in scan (e.g., based on its MAC address). In one embodiment, if the fixed device (on the wall of the facility, for example) is passive (e.g., a barcode or QR code), the machine learning system can identify this code as the check-in device.

At block 1115, the machine learning system can then determine the location of the check-in device. For example, based on a predefined mapping between device(s) and location(s) in the facility, the machine learning system can determine where the particular check-in device (determined at block 1110) is located.

At block 1120, the machine learning system can then identify the patient(s) that are associated with the determined location. For example, if the location is a private room (e.g., a residence in a long-term residential care facility), the machine learning system can determine the patient(s) that are assigned to the room. In some embodiments, if the location is in a common or shared space, the machine learning system can identify the patient(s) that were in the space at the time of the check-in (e.g., based on video, proximity sensors, patient schedules, and the like).

In this way, the machine learning system can identify which patient(s) were being assisted when the action was performed. In some embodiments, all or portions of the method 1100 may also be used to identify the relevant user(s) for the action, as discussed above. For example, using the check-in scan(s), the machine learning system can determine which user(s) engaged in the assistance.

### Example Method for Identifying Relevant User(s) and Patient(s) for Predicted Actions

FIG. 12 is a flow diagram depicting an example method for identifying relevant user(s) and/or patient(s) for predicted actions. In some embodiments, the method 1200 is performed by a machine learning system, such as machine learning system 120 of FIGS. 1 and 2. In one embodiment, the method 1200 provides additional detail for block 810 of FIG. 8, where the machine learning system identifies the relevant patient(s) for an action.

At block 1205, the machine learning system detects a user device and/or patient device via proximity data (e.g., using one or more proximity sensors). For example, as discussed above, the user(s) and/or patient(s) may carry, wear, or otherwise be associated with devices that can identify other nearby devices, identify itself to nearby devices, or both. In some embodiments, these user and patient devices act as proximity sensors to detect nearby devices. In at least embodiment, the proximity sensors may be stationary objects (in a similar manner to the check-in system described above with reference to FIG. 11).

At block 1210, the machine learning system determines whether the proximity data satisfies one or more criteria to concretely identify the patient being assisted. For example, the machine learning system may determine whether there is a patient within a defined distance from the user based on whether or not the patient and/or user are detected at all in the proximity data, how strongly the signal is detected, and the like. If the proximity data does not indicate any patient(s) within a defined distance from the user(s), the machine learning system may determine that the criteria are not satisfied. In some embodiments, if multiple patient(s) are reflected in the proximity data as within the defined distance, the machine learning system may similarly determine that the criteria are not satisfied (e.g., because it is not clear which patient was being assisted).

If the machine learning system determines that the criteria are satisfied, the method 1200 terminates at block 1220, and the identified patient is used as the relevant patient being assisted. If the machine learning system determines that the criteria are not satisfied, the method 1200 continues to block 1215.

At block 1215, the machine learning system prompts the relevant user(s) (e.g., those performing the action) to confirm which patient is being assisted, as discussed above. In some embodiments, the machine learning system can indicate a list of potential patients (e.g., those reflected in the proximity data) and allow the user to select from this reduced list. Once the patient is identified, the method 1200 terminates at block 1220.

In some embodiments, all or portions of the method 1200 may also be used to identify the relevant user(s) for the action, as discussed above. For example, using the proximity data, the machine learning system can determine which user(s) were nearby and/or engaged in the assistance.

### Example Method for Refining Machine Learning Models Based on Real-Time Motion Classifications

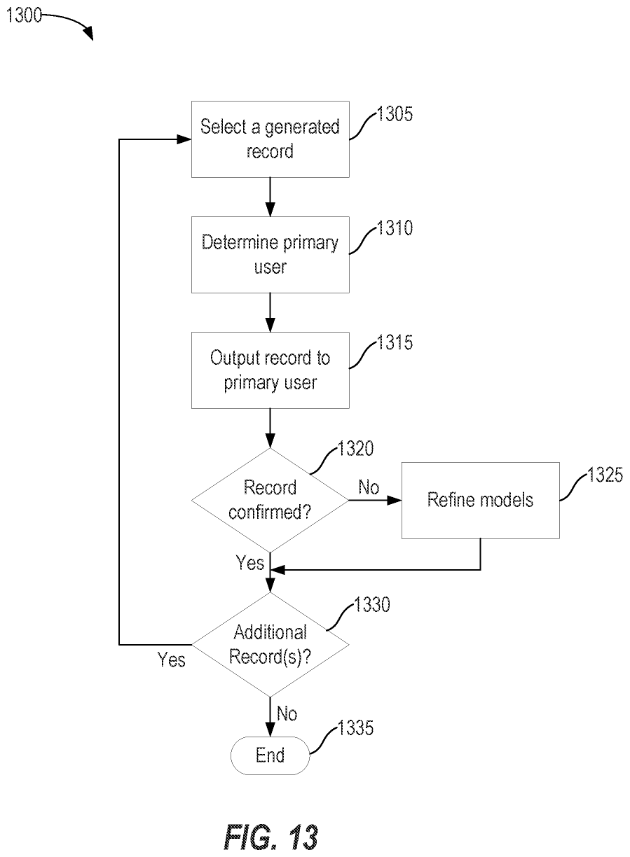

FIG. 13 is a flow diagram depicting an example method for refining machine learning models based on real-time motion classifications. In some embodiments, the method 1300 is performed by a machine learning system, such as machine learning system 120 of FIGS. 1 and 2.

In some embodiments, the method 1300 is performed for all generated records. That is, all records may be manually reviewed or verified. In some embodiments, the method 1300 is only used during initial stages of the model deployment, after training. For example, after training, the action classification models can be deployed and used to generate event records. During some initial testing phase, some or all of the generated records may be manually reviewed or verified. In one embodiment, the method 1300 is used to verify or confirm records that have one or more concerns, such as a low prediction confidence, multiple patients identified, and the like. In at least one embodiment, the method 1300 may be used to periodically check the accuracy of the model(s), such as by randomly or periodically selecting records for manual verification.

At block 1305, the machine learning system selects a generated record to be verified. As discussed above, this verification may be performed based on a variety of criteria, including verifying all automatically-generated records, randomly sampling the generated records for review, verifying records during a testing phase, verifying records where the machine learning system is not sufficiently confident, and the like. Generally, the selected record is one that was automatically generated by the machine learning system using machine learning. That is, the record was created to include an action that was identified by processing motion data using one or more machine learning models.

At block 1310, the machine learning system determines the primary user indicated in the record. As discussed above, the primary user may generally correspond to the caregiver who performed the action, the caregiver that has been assigned responsibility for the action (if multiple people performed it), and the like.

At block 1315, the machine learning system outputs the selected record to this primary user. For example, the machine learning system may transmit the event record to the user via one or more networks, such as the Internet. The event record can be output (e.g., via a graphical user interface (GUI)) to allow the user to review the details (e.g., the patient, the participating users, the time, the action(s) performed, and the like). The user can then verify that the record is accurate, or submit one or more changes.

At block 1320, the machine learning system determines whether the record was confirmed or validated by the user. If so, the method 1300 continues to block 1330. If the record was not validated (e.g., if the user indicated that one or more errors were present, or added one or more missing pieces of data, such as the specific action), the method 1300 continues to block 1325.

At block 1325, the machine learning system can optionally refine the machine learning model(s) based on the updated information. For example, suppose the user indicated that the action included in the record was incorrect, and identifies the actual action. In an embodiment, the machine learning system can use this indication as a label for the original motion data that was used to train the model(s). Subsequently, the machine learning system can use this newly-labeled data to refine the model(s), as discussed above. Although the illustrated example depicts refining the model individually for each (corrected) record for conceptual clarity, in some embodiments, the machine learning system can store the newly-labeled records for subsequent use in refining the models (e.g., during a refinement or fine-tuning phase).

The method 1300 then continues to block 1330, where the machine learning system determines whether there is at least one additional record that needs to be verified. If so, the method 1300 returns to block 1305. If not, the method 1300 terminates at block 1335.

### Example Method for Training One or More Machine Learning Models to Identify Actions

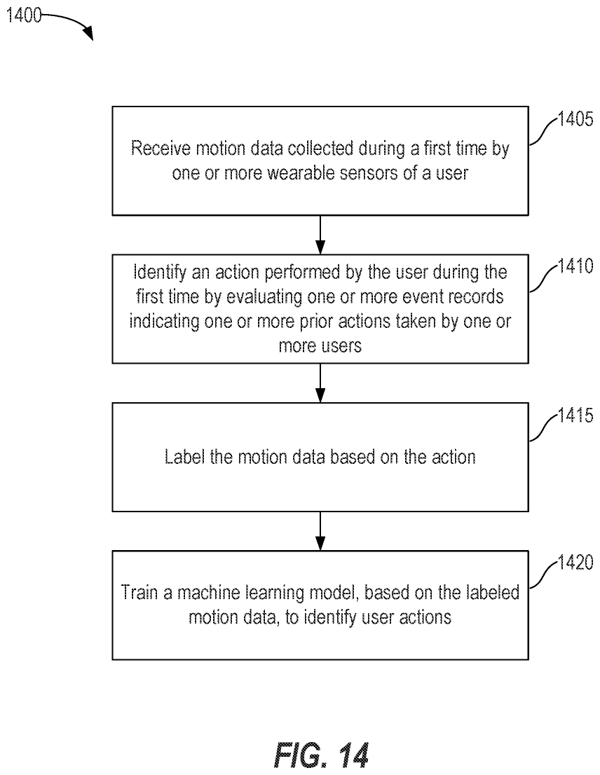

FIG. 14 is a flow diagram depicting an example method for training one or more machine learning models to identify user actions based on motion data. In some embodiments, the method 1400 is performed by a machine learning system, such as machine learning system 120 of FIGS. 1 and 2.

At block 1405, motion data collected during a first time by one or more wearable sensors of a user is received.

At block 1410, an action performed by the user during the first time is identified by evaluating one or more event records indicating one or more prior actions performed by one or more users.

At block 1415, the motion data is labeled based on the action.

At block 1420, a machine learning model is trained, based on the labeled motion data, to identify user actions.

In some embodiments, the one or more wearable sensors comprise a respective wrist-mounted sensor on each respective wrist of the user.

In some embodiments, the motion data comprises, for each respective wrist, respective accelerometer data indicating movement of the respective wrist and orientation of the respective wrist.

In some embodiments, the action corresponds to a caregiving action performed, by the user, for a patient.

### Example Method for Identifying Actions Using Machine Learning

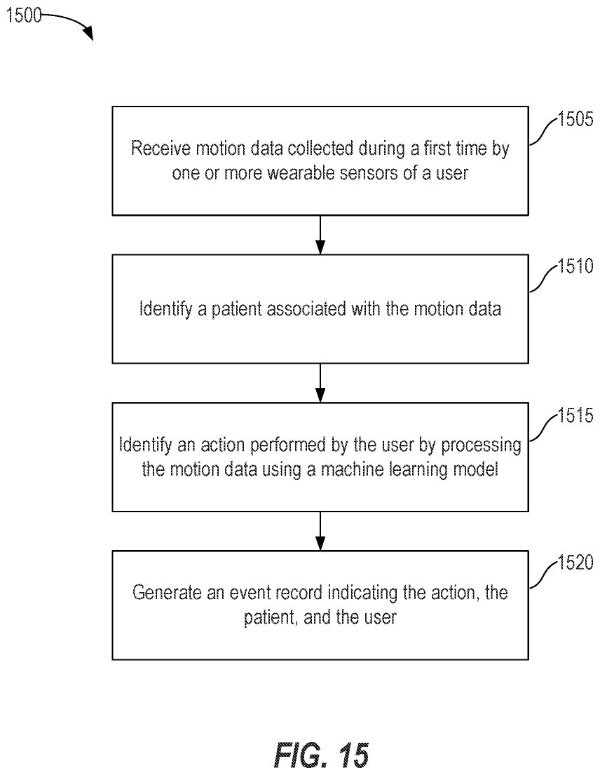

FIG. 15 is a flow diagram depicting an example method for identifying user actions using machine learning. In some embodiments, the method 1500 is performed by a machine learning system, such as machine learning system 120 of FIGS. 1 and 2.

At block 1505, motion data collected during a first time by one or more wearable sensors of a user is received.

At block 1510, a patient associated with the motion data is identified.

At block 1515, an action performed by the user is identified by processing the motion data using a machine learning model.

At block 1520, an event record indicating the action, the patient, and the user is generated.

In some embodiments, the one or more wearable sensors comprise a respective wrist-mounted sensor on each respective wrist of the user.

In some embodiments, the motion data comprises, for each respective wrist, respective accelerometer data indicating motion of the respective wrist and orientation of the respective wrist.

In some embodiments, the action corresponds to a caregiving action performed, by the user, for the patient.

In some embodiments, identifying the patient comprises determining a location of the user when the motion data was collected, and determining that the location is associated with the patient.

In some embodiments, the location of the user is determined based on a check-in scan performed by the user.

In some embodiments, the location of the user is determined using a proximity sensor.

In some embodiments, the method 1500 further includes identifying one or more other users that assisted with the action, and indicating the one or more other users in the event record.

### Example Processing System for Improved Machine Learning Models

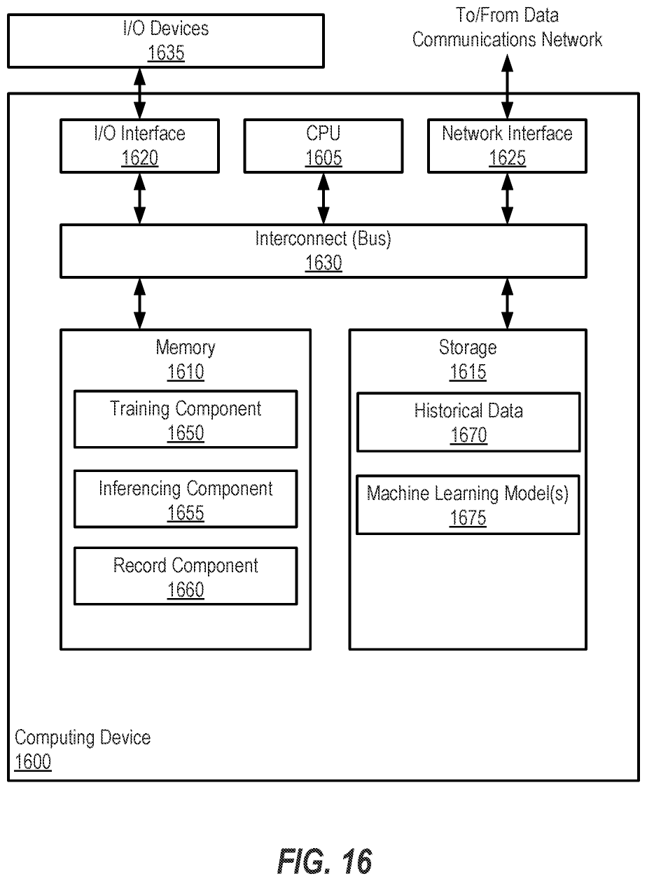

FIG. 16 depicts an example computing device 1600 configured to perform various aspects of the present disclosure. Although depicted as a physical device, in embodiments, the computing device 1600 may be implemented using virtual device(s), and/or across a number of devices (e.g., in a cloud environment). In one embodiment, the computing device 1600 corresponds to the machine learning system 120 of FIG. 1.

As illustrated, the computing device 1600 includes a CPU 1605, memory 1610, storage 1615, a network interface 1625, and one or more I/O interfaces 1620. In the illustrated embodiment, the CPU 1605 retrieves and executes programming instructions stored in memory 1610, as well as stores and retrieves application data residing in storage 1615. The CPU 1605 is generally representative of a single CPU and/or GPU, multiple CPUs and/or GPUs, a single CPU and/or GPU having multiple processing cores, and the like. The memory 1610 is generally included to be representative of a random access memory. Storage 1615 may be any combination of disk drives, flash-based storage devices, and the like, and may include fixed and/or removable storage devices, such as fixed disk drives, removable memory cards, caches, optical storage, network attached storage (NAS), or storage area networks (SAN).

In some embodiments, I/O devices 1635 (such as keyboards, monitors, etc.) are connected via the I/O interface(s) 1620. Further, via the network interface 1625, the computing device 1600 can be communicatively coupled with one or more other devices and components (e.g., via a network, which may include the Internet, local network(s), and the like). As illustrated, the CPU 1605, memory 1610, storage 1615, network interface(s) 1625, and I/O interface(s) 1620 are communicatively coupled by one or more buses 1630.

In the illustrated embodiment, the memory 1610 includes a training component 1650, an inferencing component 1655, and a record component 1660, which may perform one or more embodiments discussed above. Although depicted as discrete components for conceptual clarity, in embodiments, the operations of the depicted components (and others not illustrated) may be combined or distributed across any number of components. Further, although depicted as software residing in memory 1610, in embodiments, the operations of the depicted components (and others not illustrated) may be implemented using hardware, software, or a combination of hardware and software.

In one embodiment, the training component 1650 is used to train the machine learning model(s), such as by using the workflow 100 of FIG. 1, the workflow 200 of FIG. 2, the method 400 of FIG. 4, the method 500 of FIG. 5, the method 600 of FIG. 6, the method 700 of FIG. 7, and/or the method 1400 of FIG. 14. The inferencing component 1655 may be configured to use the models to classify motion data using trained models, such as by using the workflow 300 of FIG. 3, the method 800 of FIG. 8, the method 900 of FIG. 9, the method 1000 of FIG. 10, the method 1100 of FIG. 11, the method 1200 of FIG. 12, and/or the method 1500 of FIG. 15. The record component 1660 (or one or more other components that are not depicted) may use these classified actions to generate event records, such as by using the methods. In some embodiments, the record component 1660 can also be used to evaluate and/or verify the generated records, such as using the method 1300 of FIG. 13.

In the illustrated example, the storage 1615 includes historical data 1670 (which may correspond to labeled motion data used to train and/or evaluate the models, as well as generated event records), as well as one or more machine learning model(s) 1675. Although depicted as residing in storage 1615, the historical data 1670 and machine learning model(s) 1675 may be stored in any suitable location, including memory 1610.

### Example Clauses

Implementation examples are described in the following numbered clauses:

Clause 1: A method, comprising: receiving motion data collected during a first time by one or more wearable sensors of a user; identifying an action performed by the user during the first time by evaluating one or more event records indicating one or more prior actions performed by one or more users; labeling the motion data based on the action; and training a machine learning model, based on the labeled motion data, to identify user actions.

Clause 2: The method of Clause 1, wherein the one or more wearable sensors comprise a respective wrist-mounted sensor on each respective wrist of the user.

Clause 3: The method of any one of Clauses 1-2, wherein the motion data comprises, for each respective wrist, respective accelerometer data indicating movement of the respective wrist and orientation of the respective wrist.

Clause 4: The method of any one of Clauses 1-3, wherein the action corresponds to a caregiving action performed, by the user, for a patient.

Clause 5: A method, comprising: receiving motion data collected during a first time by one or more wearable sensors of a user; identifying a patient associated with the motion data; identifying an action performed by the user by processing the motion data using a machine learning model; and generating an event record indicating the action, the patient, and the user.

Clause 6: The method of Clause 5, wherein the one or more wearable sensors comprise a respective wrist-mounted sensor on each respective wrist of the user.

Clause 7: The method of any one of Clauses 5-6, wherein the motion data comprises, for each respective wrist, respective accelerometer data indicating motion of the respective wrist and orientation of the respective wrist.

Clause 8: The method of any one of Clauses 5-7, wherein the action corresponds to a caregiving action performed, by the user, for the patient.

Clause 9: The method of any one of Clauses 5-8, wherein identifying the patient comprises: determining a location of the user when the motion data was collected; and determining that the location is associated with the patient.

Clause 10: The method of any one of Clauses 5-9, wherein the location of the user is determined based on a check-in scan performed by the user.

Clause 11: The method of any one of Clauses 5-10, wherein the location of the user is determined using a proximity sensor.

Clause 12: The method of any one of Clauses 5-11, further comprising: identifying one or more other users that assisted with the action; and indicating the one or more other users in the event record.

Clause 13: A system, comprising: a memory comprising computer-executable instructions; and one or more processors configured to execute the computer-executable instructions and cause the processing system to perform a method in accordance with any one of Clauses 1-12.

Clause 14: A system, comprising means for performing a method in accordance with any one of Clauses 1-12.

Clause 15: A non-transitory computer-readable medium comprising computer-executable instructions that, when executed by one or more processors of a processing system, cause the processing system to perform a method in accordance with any one of Clauses 1-12.

Clause 16: A computer program product embodied on a computer-readable storage medium comprising code for performing a method in accordance with any one of Clauses 1-12.

### Additional Considerations

The preceding description is provided to enable any person skilled in the art to practice the various embodiments described herein. The examples discussed herein are not limiting of the scope, applicability, or embodiments set forth in the claims. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, the word “exemplary” means “serving as an example, instance, or illustration.” Any aspect described herein as “exemplary” is not necessarily to be construed as preferred or advantageous over other aspects.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

Embodiments of the invention may be provided to end users through a cloud computing infrastructure. Cloud computing generally refers to the provision of scalable computing resources as a service over a network. More formally, cloud computing may be defined as a computing capability that provides an abstraction between the computing resource and its underlying technical architecture (e.g., servers, storage, networks), enabling convenient, on-demand network access to a shared pool of configurable computing resources that can be rapidly provisioned and released with minimal management effort or service provider interaction. Thus, cloud computing allows a user to access virtual computing resources (e.g., storage, data, applications, and even complete virtualized computing systems) in “the cloud,” without regard for the underlying physical systems (or locations of those systems) used to provide the computing resources.

Typically, cloud computing resources are provided to a user on a pay-per-use basis, where users are charged only for the computing resources actually used (e.g. an amount of storage space consumed by a user or a number of virtualized systems instantiated by the user). A user can access any of the resources that reside in the cloud at any time, and from anywhere across the Internet. In context of the present invention, a user may access applications or systems (e.g., the machine learning system 120) or related data available in the cloud. For example, the machine learning system 120 could execute on a computing system in the cloud and train and/or use machine learning models. In such a case, the machine learning system 120 could train models to classify user actions, and store the models at a storage location in the cloud. Doing so allows a user to access this information from any computing system attached to a network connected to the cloud (e.g., the Internet).

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A method of training machine learning models, comprising:
receiving motion data collected during a first time by one or more wearable sensors of a user;
identifying an action performed by the user during the first time by evaluating one or more event records indicating one or more prior actions performed by one or more users;
labeling the motion data based on the action; and
training a machine learning model, based on the labeled motion data, to identify user actions.

2. The method of claim 1, wherein the one or more wearable sensors comprise a respective wrist-mounted sensor on each respective wrist of the user.

3. The method of claim 2, wherein the motion data comprises, for each respective wrist, respective accelerometer data indicating movement of the respective wrist and orientation of the respective wrist.

4. The method of claim 1, wherein the action corresponds to a caregiving action performed, by the user, for a patient.

5. A method of classifying motion using machine learning, comprising:
receiving motion data collected during a first time by one or more wearable sensors of a user;
identifying a patient associated with the motion data;
identifying an action performed by the user by processing the motion data using a machine learning model; and
generating an event record indicating the action, the patient, and the user.

6. The method of claim 5, wherein the one or more wearable sensors comprise a respective wrist-mounted sensor on each respective wrist of the user.

7. The method of claim 6, wherein the motion data comprises, for each respective wrist, respective accelerometer data indicating motion of the respective wrist and orientation of the respective wrist.

8. The method of claim 5, wherein the action corresponds to a caregiving action performed, by the user, for the patient.

9. The method of claim 5, wherein identifying the patient comprises:
determining a location of the user when the motion data was collected; and
determining that the location is associated with the patient.

10. The method of claim 9, wherein the location of the user is determined based on a check-in scan performed by the user.

11. The method of claim 9, wherein the location of the user is determined using a proximity sensor.

12. The method of claim 5, further comprising:
identifying one or more other users that assisted with the action; and
indicating the one or more other users in the event record.

13. A non-transitory computer-readable storage medium comprising computer-readable program code that, when executed using one or more computer processors, performs an operation for classifying motion using machine learning comprising:
receiving motion data collected during a first time by one or more wearable sensors of a user;
identifying a patient associated with the motion data;
identifying an action performed by the user by processing the motion data using a machine learning model; and
generating an event record indicating the action, the patient, and the user.

14. The non-transitory computer-readable storage medium of claim 13, wherein the one or more wearable sensors comprise a respective wrist-mounted sensor on each respective wrist of the user.

15. The non-transitory computer-readable storage medium of claim 14, wherein the motion data comprises, for each respective wrist, respective accelerometer data indicating motion of the respective wrist and orientation of the respective wrist.

16. The non-transitory computer-readable storage medium of claim 13, wherein the action corresponds to a caregiving action performed, by the user, for the patient.

17. The non-transitory computer-readable storage medium of claim 13, wherein identifying the patient comprises:
determining a location of the user when the motion data was collected; and
determining that the location is associated with the patient.

18. The non-transitory computer-readable storage medium of claim 17, wherein the location of the user is determined based on a check-in scan performed by the user.

19. The non-transitory computer-readable storage medium of claim 17, wherein the location of the user is determined using a proximity sensor.

20. The non-transitory computer-readable storage medium of claim 13, the operation further comprising:
identifying one or more other users that assisted with the action; and
indicating the one or more other users in the event record.
